(19) United States
(12) Patent Application Publication
(10) Pub. No.: US 2010/0004187 A1
(43) Pub. Date: Jan. 7, 2010

(54) COBALT-AMINE BASED METAL COMPLEX AS AN ANTIBACTERIAL COMPOUND

(75) Inventors: Eddie L. Chang, Silver Spring, MD (US); Daniel Zabetakis, Brandywine, MD (US); Andy Knight, New Orleans, LA (US); Monique L. van Hoek, Centreville, VA (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(73) Assignee: The Government of the United States of America, as represented by the Secretary of the Navy, Washington, DC (US)

(21) Appl. No.: 12/494,750

(22) Filed: Jun. 30, 2009

Related U.S. Application Data
(60) Provisional application No. 61/076,902, filed on Jun. 30, 2008.

(51) Int. Cl. A61K 31/295; A61K 31/65; A61K 31/7048; A61P 31/04

(52) U.S. Cl. 514/27; 514/501; 514/154

(57) ABSTRACT

Disclosed herein is a method of: administering to a subject suspected of being infected with a bacterium, a composition of a cobalt (III) compound having the formula CoR¹R²R³R⁴R⁵R⁶ or a salt thereof and an antibiotic compound. Each of R¹, R², R³, R⁴, and R⁵ is the same or different and includes an N-based ligand donor atom selected from the group consisting of ammonia, primary amine, or secondary amine, or salt thereof. R⁶ is a ligand. Also disclosed herein is a composition of the above cobalt (III) compound and an antibiotic compound. Also disclosed herein is a method of administering the above cobalt (III) compound to a subject diagnosed as needing a broad spectrum antibiotic

This application claims the benefit of U.S. Provisional Application No. 61/076,902 filed on Jun. 30, 2008. The provisional application and all other publications and patent documents referred to throughout this nonprovisional application are incorporated herein by reference.

### TECHNICAL FIELD

The present disclosure is generally related to a class of antibacterial compounds.

### DESCRIPTION OF RELATED ART

Co(NH₃)₆³⁺ (hereinafter “Cohex”) is an octahedral-coordinated Co(III) ion complex that is also a biological mimic of the hexahydrate Mg(II) ion, [Mg(H₂O)₆]²⁺ and can bind to critical microbial components that either facilitate the transport of the magnesium ion or the efflux of drugs or virulence factors. Cohex has also been shown to have antiviral properties (US Patent Application Publication No. 2008/0182835).

There is a recent report of another kind of cobalt complex, based on coordination with pyridine-amide ligands, that shows antibacterial activity (Mishra et al., Synthesis, characterization and antibacterial activity of cobalt(III) complexes with pyridine-amide ligands. European Journal of Medicinal Chemistry 2007, 43(10), 2189-2196). These complexes consist of Co(III) ions surrounded by either three bidentate ligands or two tridentate ligands. Two variants of these complexes showed activity against standard and pathogenic resistant bacteria. It is not known how these compounds work against the bacteria, but it is believed that the uncoordinated pyridine rings of the ligand may be interacting with additional metal ions.

### BRIEF SUMMARY

Disclosed herein is a method comprising: administering to a subject suspected of being infected with a bacterium, a composition comprising a cobalt (III) compound having the formula CoR¹R²R³R⁴R⁵R⁶ or a salt thereof and an antibiotic compound. Each of R¹, R², R³, R⁴, and R⁵ is the same or different and includes an N-based ligand donor atom selected from the group consisting of ammonia, primary amine, or secondary amine, or salt thereof. R⁶ is a ligand.

Also disclosed herein is a composition comprising the above cobalt (III) compound and an antibiotic compound.

Also disclosed herein is a method comprising administering the above cobalt (III) compound to a subject diagnosed as needing a broad spectrum antibiotic.

### BRIEF DESCRIPTION OF THE DRAWINGS

A more complete appreciation of the invention will be readily obtained by reference to the following Description of the Example Embodiments and the accompanying drawings.

FIG. 1 shows cobalt (III) hexammine, Co(NH₃)₆³⁺ and magnesium (II) hexahydrate, Mg(H₂O)₆²⁺, both forming octahedral coordination geometries with their respective ligands

FIG. 2 shows the structure of TolC and its periplasmic entrance. Cohex binds near the periplasmic opening, in the constriction of the entrance. (after Anderson, C., et al, Proc. Natl. Acad. Sci., USA, 99, 11103 (2002))

FIG. 3 shows an electrostatic view of CorA (Thermotoga maritime). Positively charged residues are blue and negatively charged or hydroxyl-containing residues are red. Note the red region in the interior of the funnel region (from Lunin, et al, 2006).

FIG. 4 shows F. tularensis subspecies Novicida % inhibition plotted against log (Cohex(mM)). 1 and 2 refer to different runs.

FIG. 5 shows F. philomiragia % inhibition plotted against log(Cohex(mM)). 1 and 2 refer to different runs.

FIG. 6 shows the cytotoxicity of Cohex plotted as % Viable BHK cells against log(Cohex).

FIG. 7 shows the interaction Cohex with kanamycin.

FIG. 8 shows the Cohex interaction with streptomycin.

FIG. 9 shows the Cohex interaction with tetracycline.

FIG. 10 shows the Cohex interaction with gentamicin.

FIG. 11 shows an agar dish with 1.0 (10, TC), 2.0 (20, TC) mg of tetracycline apposed to 0.78 (10, CH) and 1.6 (20, CH) mg Cohex; the zones of clearance is outlined in black line, show that they abut each other for the “20” disks. The dotted circle shows an additional clearance zone associated with Cohex.

### DETAILED DESCRIPTION OF EXAMPLE EMBODIMENTS

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

Described herein is the use of Cohex as an antibiotic. The concept is based upon previously observed blockage of the export ports of bacteria (Higgins et al., Structure of the Ligand-blocked Periplasmic Entrance of the Bacterial Multidrug Efflux Protein TolC. J. Mol. Biol. 2004, 342, 697-702), the inhibition of bacterial Mg²⁺ transporters (Kucharski et al., Cation hexaammines are selective and potent inhibitors of the CorA magnesium transport system. Journal of Biological Chemistry 2000, 275, (22), 16767-16773; Lunin et al., Crystal structure of the CorA Mg2+ transporter. Nature 2006, 440, (7085), 833-837), and observations of the antibiotic properties of Cohex. A possible basis for Cohex activity is its many similarities to hydrated Mg²⁺ ions, which allows it to preempt some of the magnesium ions' functions. In addition, because the use of Mg²⁺ ions by bacteria is ubiquitous, Cohex should also have a potential as a broad spectrum antibiotic. Furthermore, since the export port of bacteria is an important mode by which bacteria acquires resistance to certain antibiotics, the blockade of the export ports of bacteria by Cohex points to a potential application the compound can play in decreasing bacterial toxicity, in enhancing antibiotic activity, and in enabling older antibiotics to be active again against previously resistant strains of bacteria.

In this way, Cohex can act as either an antibiotic, per se, or as an “adjuvant/enhancer” drug that could both decreases the virulence of the bacterium while allowing other drugs to act with better effect.

The basis of this approach lies in the observations that Cohex inhibits Mg(II) transport for CorA (Kucharski) from E. coli and that the metal complex binds strongly to the periplasmic entrance of a TolC ortholog in E. coli (Higgins). Because CorA and its analogs are ubiquitous in eubacterial and archaeal microorganisms (Kucharski; Lunin; Smith et al., Microbial Magnesium transport: unusual transporters searching for identity. Molecular Microbiology 1998, 28, 217-226; Szegedy et al., The CorA Mg²⁺ Transport Protein of Salmonella typhimurium. MUTAGENESIS OF CONSERVED RESIDUES IN THE SECOND MEMBRANE DOMAIN. J. Biol. Chem. 1999, 274, (52), 36973-36979) and the TolC family of envelope proteins is found throughout Gram-negative bacteria (Higgins; Gil et al., Deletion of TolC orthologs in Francisella tularensis identifies roles in multidrug resistance and virulence. PNAS % R 10.1073/pnas.0602582103 2006, 103, (34), 12897-12902; Koronakis et al., Crystal structure of the bacterial membrane protein TolC central to multidrug efflux and protein export. Nature 2000, 405, (6789), 914-919), Cohex has the potential to be a wide-ranging, broad-spectrum, anti-microbial therapeutic that can also decrease virulence. It is further likely that the antibacterial properties of Cohex can be enhanced when used in combination with other antibiotics as part of a rational approach to drug development (von Itzstein et al., Rational design of potent sialidase-based inhibitors of influenza virus replication. Nature 1993, 363, (6428), 418-423). Added advantages of Cohex are that the compound has also shown broad spectrum antiviral activity (Delehanty et al, Antiviral Properties of Cobalt(III)-Complexes, Bioorganic &Amp; Medicinal Chemistry, 16 (2) 830-837 (2008)), that it can be made in large quantities cost-effectively, and is very stable. Thus, Cohex may be one of very few compounds that is not highly toxic and has both antiviral and antibacterial properties.

### Biological Mg²⁺ and Cohex

The chemistry of Mg²⁺(aq) is highly unusual among biologically relevant cations. Magnesium (II) in solution (Mg(H₂O)₆²⁺(aq)) has the largest hydrated radius, smallest ionic radius, and greatest charge density when compared to Ca, K, and Na ions. Water molecules of hydration are also bonded 3-4 orders of magnitude more tightly to Mg²⁺ than to other biological cations and its volume of the hydrated cation is almost 400 fold that of the atomic cation. Interaction of Mg(H₂O)₆²⁺with other biomolecules can be through direct, inner-sphere coordination to a ligand or through indirect outer-sphere complexation (e.g., as a cofactor in exonuclease III, Rnase H, and hairpin ribozymes). It has been known for some time that certain metal ions, such as Cr(III) and Co(III) can be used in place of magnesium for studying its biological activity. For example, the feasibility of using chromium or cobalt nucleotides (e.g., CrATP, Co(NH₃)₄ATP) as substrates for kinases has been demonstrated (Cleland et al., Chromium (III) and Cobalt (III) nucleotides as biological probes. Advances in Inorganic Biochemistry 1979, 1, 163-191) and Co(III) complexes have been used in place of Mg²⁺(aq) to study the role Mg²⁺(aq) plays in the catalytic mechanism of enzymes and in RNA folding (Cowan, J. A., Metallobiochemistry of Magnesium—Coordination-Complexes with Biological Substrates—Site Specificity, Kinetics and Thermodynamics of Binding, and Implications for Activity. Inorganic Chemistry 1991, 30, (13), 2740-2747; Cowan, J. A., Coordination Chemistry of Mg²⁺ and 5s Ribosomal-RNA (Escherichia-Coli)—Binding Parameters, Ligand Symmetry, and Implications for Activity. Journal of the American Chemical Society 1991, 113, (2), 675-676; Cowan, J. A., Metallobiochemistry of RNA—Co(NH₃)₆³⁺ as a Probe for Mg²⁺(Aq) Binding-Sites. Journal of Inorganic Biochemistry 1993, 49, (3), 171-175; Jou et al., Ribonuclease-H Activation by Inert Transition-Metal Complexes—Mechanistic Probes for Metallocofactors—Insights on the Metallobiochemistry of Divalent Magnesium-Ion. Journal of the American Chemical Society 1991, 113, (17), 6685-6686).

These substitutions, especially when using Co(NH₃)₆³⁺, work best when the mechanism of interaction involves either fully or partially hydrated magnesium (II) ions, where both outer-sphere complexation and electrostatic attraction can play a role. Mg(H₂O)₆²⁺(aq) and Cohex both form octahedral-coordinated inner-sphere ligands (water as ligands in former case and NH₃ in the latter, see FIG. 1), they are similar in size, both have a high charge density, and Cohex can form an outer-sphere H-bond network similar to that formed by Mg(H₂O)₆²⁺(aq) (Jou). The major differences are that Co(III) is triply charged and any exchange of inner-sphere ligands is very slow, due to the kinetic inertness of Co(III). These differences, particularly the kinetic inertness, are of interest to researchers who wish to separate inner-sphere and outer-sphere interactions of Mg(II) with its various substrates. The differences in charge and kinetic inertness between Mg(H₂O)₆²⁺(aq) and Cohex are particularly relevant to the proposed project. While the similarities between these complexes permit Cohex to bind to Mg(II) sites in bacterial proteins, the afore mentioned differences will allow Cohex to effectively “poison” these sites. Therefore, the present method may capitalize on both the high (+) charge-density of Cohex and its similarity to Mg(II) ions as ways to weaken the target bacteria.

### The TolC Target Protein

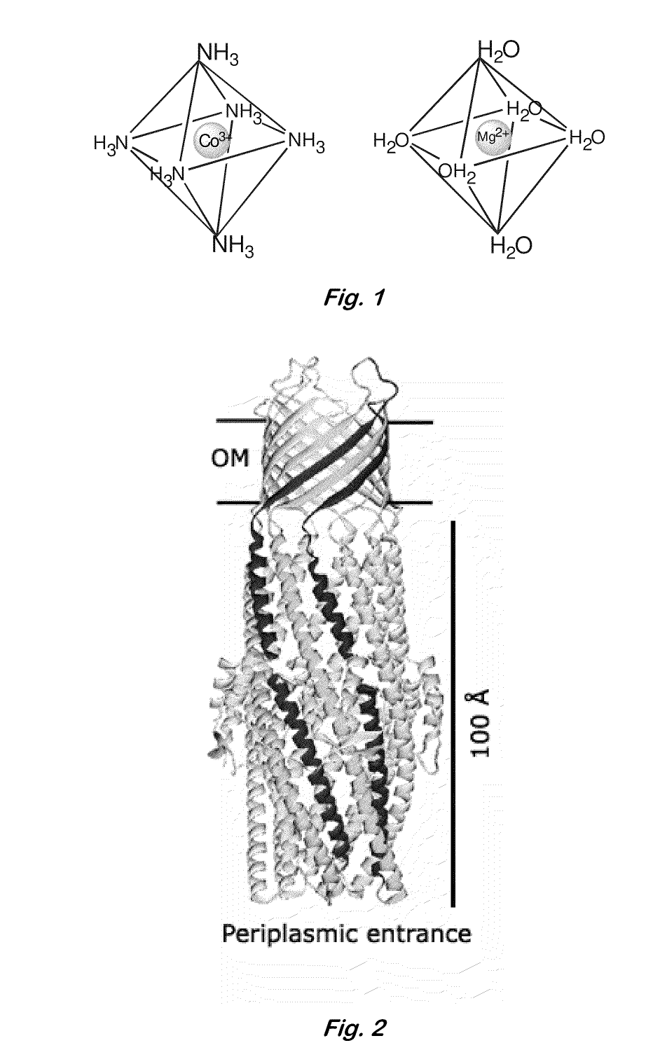

An example of a potential major target for Cohex is the bacterial TolC protein. TolC is a common outer membrane (OM) channel in Gram-negative bacteria and is used in both type I secretion systems and the export of large proteins, proteases, and small noxious compounds, most notably antibacterial drugs (Gil). The trimeric TolC forms a single pore that comprises a 100 Å long α-helical barrel projecting across the periplasmic space, and is anchored to the outer membrane by a 40 Å long β-barrel (FIG. 2) (Koronakis).

Since so many types of molecules, ranging from small antibacterial drugs to large protein toxins, are exported directly across the cell membranes of Gram-negative bacteria with the help of TolC channels, they are important components in determining both pathogenicity and drug resistance. Loss of TolC function, or the function of one of its orthologs, impacts bacterial survival and attenuates virulence (Gil; Koronakis; Andersen et al., Transition to the open state of the TolC periplasmic tunnel entrance. Proc Natl Acad Sci USA. 2002, 99, (17), 11103-11108). A recent study showed that deletion of either of the two TolC orthologs (tolC and ftlC) of F. tularensis increased sensitivity to various antibiotics, detergents, and dyes and resulted in significant attenuation of virulence in a tularemia mouse model (Gil).

TolC (E. coli) binds Mg²⁺ and the conductance of these TolC pores reconstituted in black lipid bilayers has been reported to be severely inhibited by the addition (nM amounts) of divalent and trivalent cations (Andersen et al., An aspartate ring at the TolC tunnel entrance determines ion selectivity and presents a target for blocking by large cations. Mol. Microbiol. 2002, 44, 1131-1139). Isothermal titration calorimetry data indicates that ˜1 Cohex ion is bound per trimeric TolC with a binding affinity of 20 nM, which is consistent with the results observed in the conductance inhibition studies (Higgins). Crystal structure analysis to 2.75 Å resolution showed that Cohex most likely lies in the constriction in the periplasmic entrance of TolC where it is coordinated solely by the side chains of three Asp residues (Asp³⁷⁴ from each polypeptide chain). It is interesting to note that the authors were not able to confirm the effect of Cohex on TolC with in vivo assays because the compound prevents bacterial growth at micromolar concentrations (Higgins).

### CorA Mg²⁺ Transporters

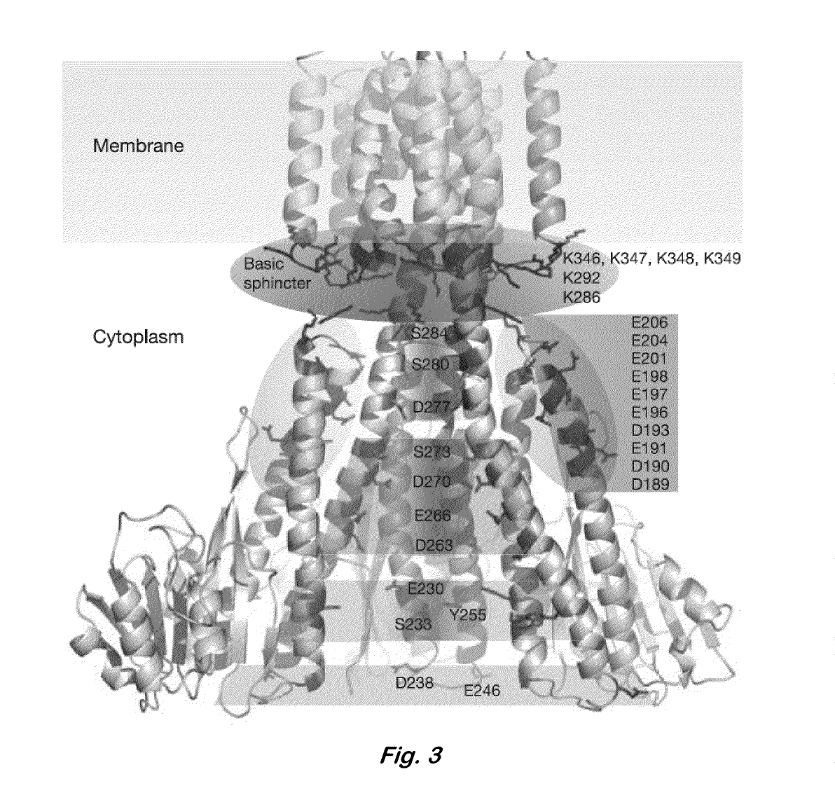

An even more specific potential target for Cohex as a Mg²⁺ mimic are the CorA Mg²⁺ transporters, which are ubiquitous in bacteria and archaea and are the major mode of magnesium uptake by these microorganisms. The CorA topology consists of a large N-terminal periplasmic domain with a smaller multi-segmented transmembrane domain (Smith et al., Sequence and topology of the CorA magnesium transport systems of Salmonella typhimurium and Escherichia coli. Identification of a new class of transport protein. J. Biol. Chem. 1993, 268, (19), 14071-14080). A recent crystal structure reveals CorA to be a homopentamer with the periplasmic portion in the shape of a funnel (FIG. 3) (Lunin) and whose interior is lined with negatively charged or hydroxyl-containing residues. The large periplasmic domain of CorA, then, provides a natural binding site for cations that can subsequently interact with the membrane domain. Uniquely, CorA does not appear to utilize electrostatic interactions to move Mg²⁺ across the membrane because there is only one negatively charged residue in the channel.

The uptake of ⁶³Ni²⁺ by CorA of both Salmonella typhimurium and Methanococcus jannaschii was reported to be inhibited by Cohex and other Mg²⁺(aq) analogs, such as Ru(II) and Ru(III)hex (Kucharski), even though the M. jannaschii CorA shares 38% sequence homology (16% identity) to that of S. typhimurium. Cohex had an IC50 of 1 mM, corresponding to a Kᵢ of 500 nM. The chloropentammines of Co(III) and Ru(III) also inhibited Ni uptake, but were not as potent as the hexammine cations.

Cohex also inhibited the growth of a S. typhimurium strain in which CorA was the only functional Mg²⁺ transporter. Atomic absorption showed that Cohex likely inhibited Mg uptake without themselves being transported into the cytosol. Cohex does not inhibit the alternate pathways of Mg²⁺ transport such as those mediated by MgtB Mg transporter. It has therefore been inferred that the initial Mg binding site on CorA is specific for a hydrated Mg²⁺ (hence its susceptibility to Cohex), and the non-affected transporters, e.g. MgtB, probably bind a partially stripped Mg ion.

The composition may be administered to a subject in any amount and by any means that produces a therapeutic or prophylactic effect. The subject may have been diagnosed as needing a broad spectrum antibiotic.

Suitable cobalt (III) compounds include, but are not limited to, Co(NH₃)₆³⁺, CoCl(NH₃)₅²⁺, and salts thereof. The compound may also be combined with an antibiotic such as, but not limited to, tetracycline, streptomycin, kanamycin, and gentamicin. Other suitable antibiotics include, but are not limited to, amikacin, neomycin, netilmicin, tobramycin, paromomycin, geldanamycin, herbimycin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibute, ceftizoxime, ceftriaxone, cefepime, ceftpbiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, grepafloxacin, sparfloxacin, mafenide, prontosil, sulfacetamide, sulfamethizole, sulfanilimide, sulfasalazine, sulfisoxazole, trimethoprim, trimethoprim-sulfamethoxazole, demeclocycline, doxycycline, minocycline, oxytetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin, dalfopristin, rifampin, thiamphenicol, and tinidazole. The combination may have a greater antibacterial or bacteriostatic effect than either compound alone. The compound may be useful in treating infections by E. coli or bacteria containing a TolC protein or a CorA protein.

The following examples are given to illustrate specific applications. These specific examples are not intended to limit the scope of the disclosure in this application.

### Example 1

Preparation of Cobalt complexes—The synthesis of Cohex is fairly straight forward, using air to oxidize Co(II) to Co(III):

CoCl₂+4NH₄Cl+20NH₃+O₂→4[Co(NH₃)₆]Cl₃+2H₂O

9.6 g of CoCl₂.6H₂O (0.06 mol) and 6.4 g of NH₄Cl (0.12 mol) were added to 40 mL of water in a 250 mL Erlenmeyer flask with a side arm and shaken until most of the salts were dissolved. Then 1 g of fresh activated decolorizing charcoal and 20 mL concentrated ammonia were added. Next the flask was connected to the aspirator or vacuum line and air drawn through the mixture until the red solution became yellowish brown (usually 2-3 hours). The air inlet tube should be of fairly large bore (˜10 mm) to prevent clogging with the precipitated Co(NH₃)₆³⁺ salt.

The crystals and charcoal were filtered on a Büchner funnel and then a solution of 6 mL of concentrated HCl in 75 mL of water was added. The mixture was heated on a hot plate to effect complete solution and then filtered hot. The hexamminecobalt (III) chloride was crystallized by cooling to 0° C. and by slowly adding 15 mL of concentrated HCl. The crystals were filtered, washed with 60% and then 95% ethanol, and dried at 80-100° C.

### Example 2

96 well/microtiter plate assay—Assays were performed in sterile round bottom 96 well plates using Trypticase Soy Broth plus 0.1% cysteine media or Luria Broth. Media was added to control wells, along with the appropriate dilutions of Cohex, to be used as background subtraction after data collection. Triplicate rows of bacteria (100 μL of 1×10⁵ CFU/mL bacteria to each well) were added to test wells along with the serially diluted Cohex, and incubated 18-24 hours before optical reading (600 nm).

### Example 3

Cell Culture—Baby hamster kidney (BHK) cells were cultured as exponentially growing subconfluent monolayers in complete growth medium (DMEM supplemented with 1% (v/v) antibiotic/antimycotic and 10% (v/v) heat inactivated fetal bovine serum (FBS)). Cells were grown in either T25 or T75 flasks (Corning) and incubated at 37° C. under 5% CO₂ atmosphere. A subculture was performed every 3-4 days.

J774A.1 murine macrophages were cultured in monolayers in DMEM plus 10% FBS. Cells were grown in either T25 or T75 flasks (Corning) and incubated at 37° C. under 5% CO₂ atmosphere. A subculture was performed every 3-4 days. Cells were infected with Francisella species or Francisella mutants and then treated with Cohex at varying concentrations.

Francisella tularensis subspecies Novicida and Francisella philomiragia (1×10⁺⁵ CFU/mL original concentration; 5×10⁺⁴ CFU/mL final concentration in wells) were mixed with serially diluted Cohex concentration, from 0 to 2,000 mM. The density of the bacterial suspension was measured optically and a percent inhibition calculated using the formula:

$$100 * ( 1 - \frac{T ( Cohex )}{T ( Cohex = 0 )} )$$

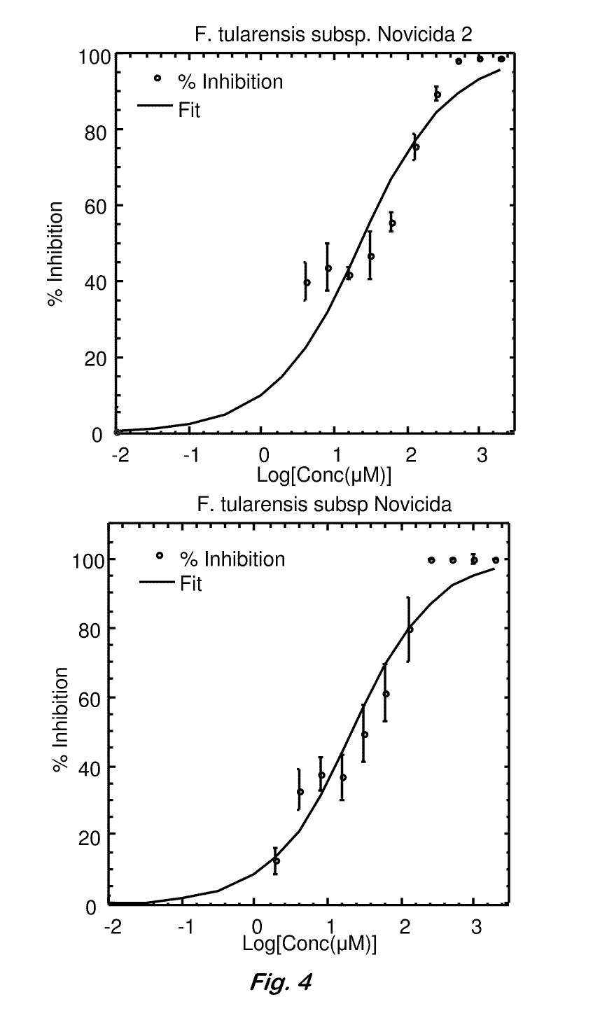

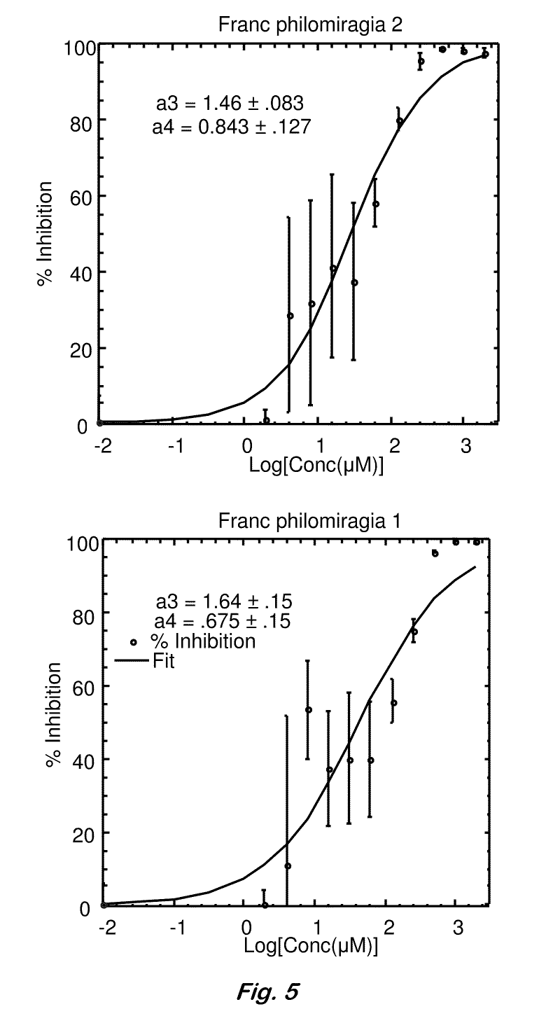

Plots of the log(Cohex(mM)) are shown in FIGS. 4 and 5 and the IC50 values given in Table 1.

TABLE 1
IC50 values (mM) obtained from the % inhibition assays

| | F. tularensis subsp Novicida | F. philomiragia |
|---|---|---|
| Assay 1 | 21.4 ± 1.2 mM | 43.6 ± 1.4 mM |
| Assay 2 | 22.9 ± 1.3 mM | 28.8 ± 1.2 mM |

It was found that the inhibition of the bacterial growth was bacteriostatic rather than bacteriocidal. The micromolar range of efficacy of Cohex indicates an efficient antibiotic against both strains of Francisella.

### Example 4

Cytotoxicity of cobalt(III) compounds—The in vitro toxicity of the cobalt(III) compounds was determined using the CellTiter96® Cell Proliferation Assay (Promega), a quantitative colorimetric assay based upon the conversion of a tetrazolium salt substrate into a blue formazan product by viable cells. At the assay endpoint, the absorbance at 570 nm is directly proportional to the number of viable cells. BHK cells were seeded into the wells of a 96-well tissue culture microtiter plate (2×10⁴ cells/well) and cultured overnight at 37° C. in a humidified atmosphere containing 5% CO₂. The next day, the compounds were serially diluted into tissue culture media and incubated with the cells for 72 hrs. For all compounds, triplicate wells were included for each concentration. At the end of the 72 hour culture period, 15 μL of tetrazolium substrate was added to each well and the plate was returned to the incubator for 4 hours to allow viable cells to convert the substrate into the formazan product. Subsequently, 100 μL of solubilization solution was added to each well and the plate was incubated overnight. The absorbance values for each concentration were divided by the absorbance values from wells with cells cultured in the absence of compound (control wells corresponding to maximum color formation). To control for any absorbance due to the compounds themselves, a second set of control wells containing no cells was included for each compound concentration.

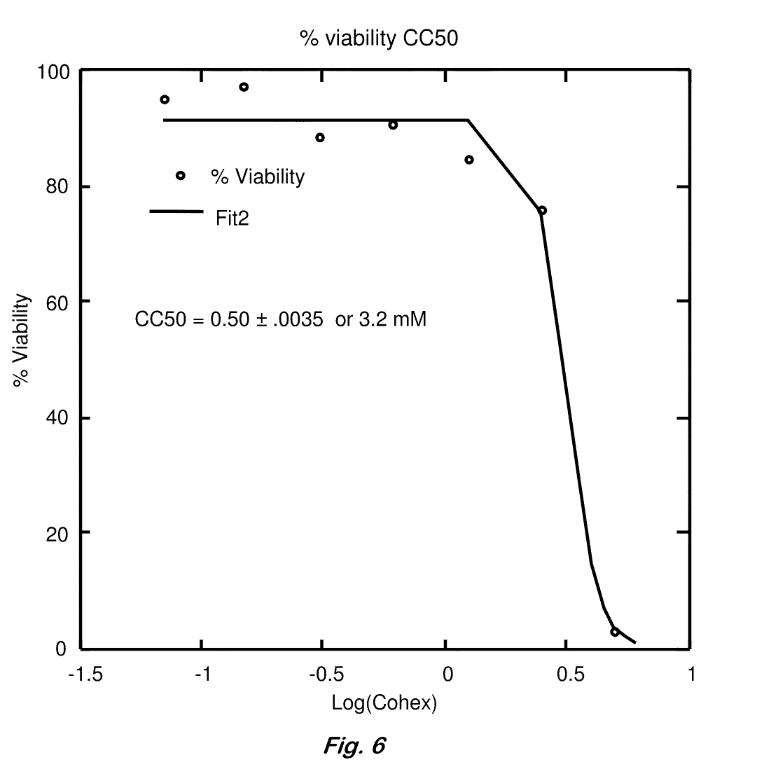

Cohex was found not to be highly toxic to mice or cytotoxic. Studies of its toxicity against BHK cells showed a CC50 of about 3.2 mM (3,200 mM) Cohex (FIG. 6).

### Example 5

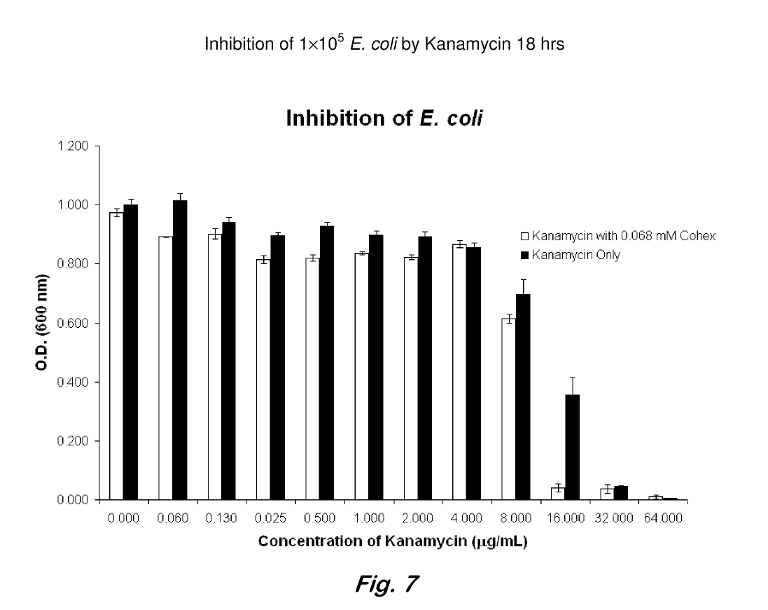

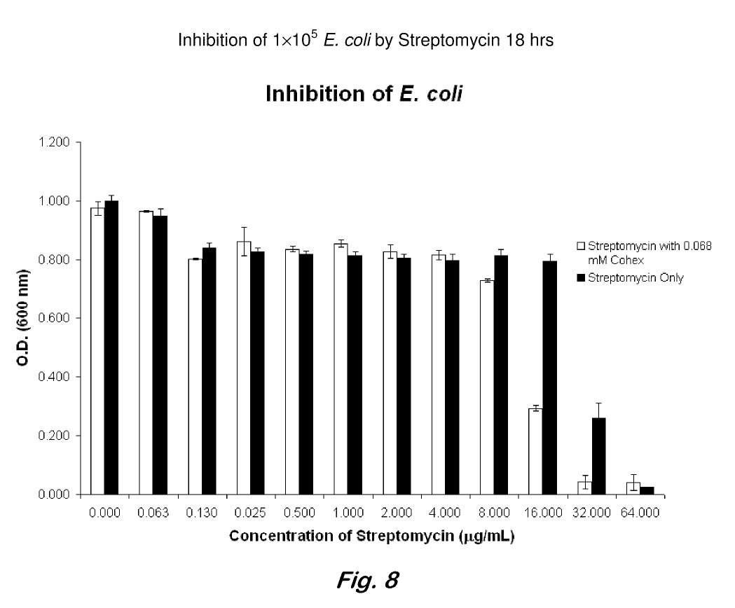

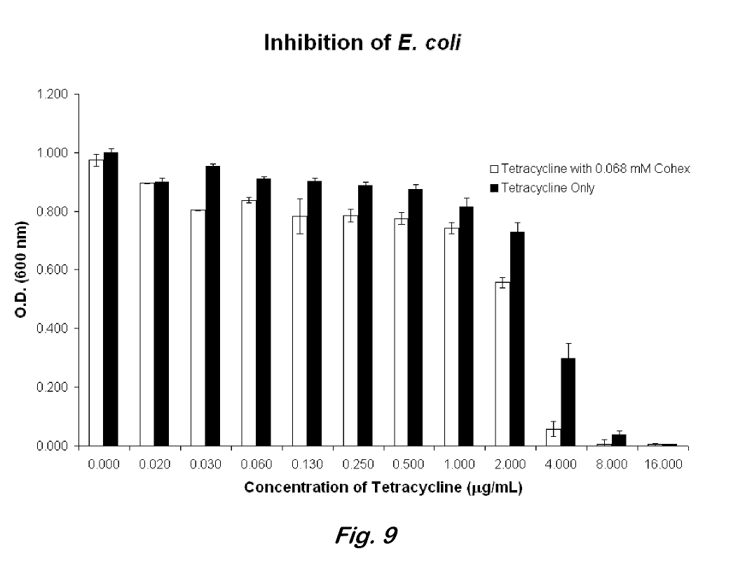

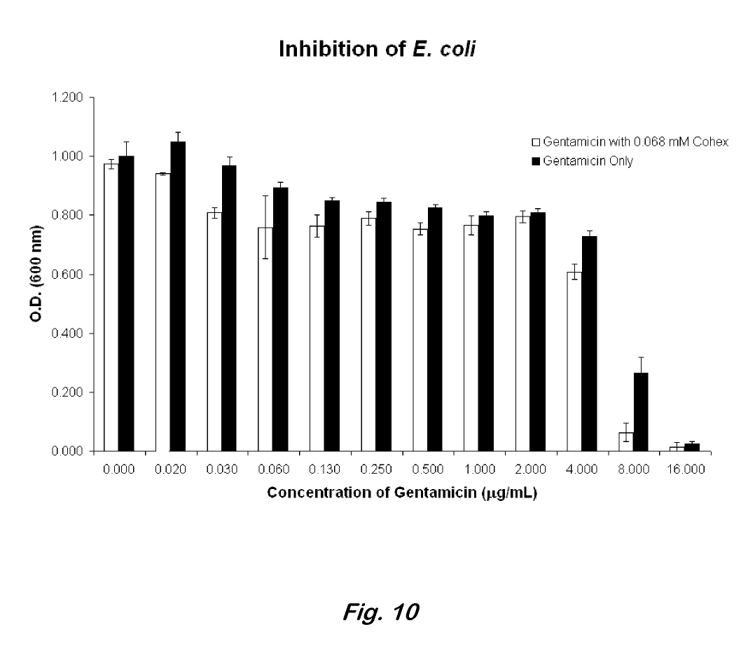

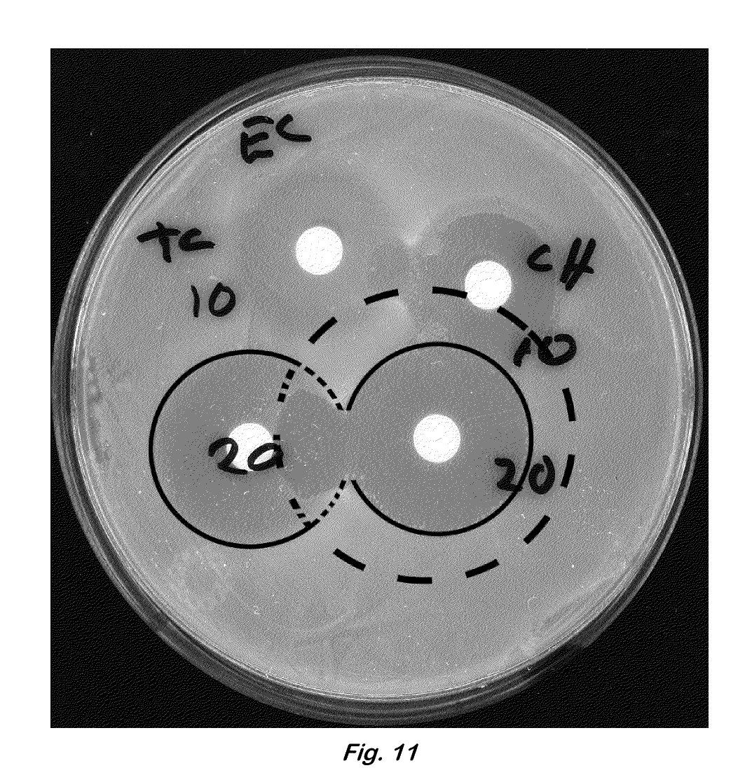

Optical density—Assays of the effect of Cohex and antibiotics was done to determine if there is an additive effect. Results show that at Cohex concentrations of 0.068 mM (a concentration at which Cohex has no effect on bacterial growth) in combination with kanamycin at a concentration of 16 μg/mL the optical density at 600 nm decreased from 0.35 to 0.040 (FIG. 7). For streptomycin at a concentration of 16 μg/mL the optical density at 600 nm decreased from 0.80 to 0.30 (FIG. 8). For tetracycline at a concentration of 4 μg/mL the optical density at 600 nm decreased from 0.30 to 0.05 (FIG. 9) and for gentamicin at a concentration of 8 μg/mL the optical density at 600 nm decreased from 0.27 to 0.07 (FIG. 10). That is, the presence of Cohex increased the potency of the drugs up to about an order of magnitude.

With both IC50 and CC50 known, it can be estimated how selective Cohex is in inhibiting Francisella growth over killing of mammalian cells by taking the ratio of CC50/IC50. Using the average of the two IC50s for each strain, it was estimated that the in vitro “selectivity ratio” of Cohex is 145 and 88 for Novicida and philomiragia, respectively. These selectivity numbers compare well with drugs that have low therapeutic ratios (e.g. lithium carbonate), typically around 2.

### Example 6

Diffusion disk assay—Diffusion disks infused with 1.0 (10, TC) and 2.0 (20, TC) mg of tetracycline (tet) were placed apposed on a nutrient agar (Difco) plate in a dish, with a lawn of E. coli, to disks infused with 0.78 (10, CH) and 1.6 (20, CH) mg of Cohex, respectively. These filters were added to the plate and incubated overnight at 37° C. The next day the plate was examined for areas of suppressed bacterial growth indicating sensitivity to the chemicals on the filters.

An extended zone of inhibition was observed for both the 1.0 tet/0.78 Cohex and the 2.0 tet/1.6 mg Cohex pairings, indicating a synergistic effect (FIG. 9). The black circles, which abut each other, outline the zones of clearance for both tet and Cohex. An additional odd-shaped clearance zone is seen around the tet disk. The shape is consistent with the added efficacy of Cohex, in the presence of tet (indicated by the dotted circle and dotted intersection region). The added clearance region is one where the concentration of Cohex is less that required for clearance by Cohex it self, but where at least the minimum concentration of tet is present. This effect is also observed, albeit to a lesser extent, for the less concentrated disks.

Obviously, many modifications and variations are possible in light of the above teachings. It is therefore to be understood that the claimed subject matter may be practiced otherwise than as specifically described. Any reference to claim elements in the singular, e.g., using the articles “a,” “an,” “the,” or “said” is not construed as limiting the element to the singular.

What is claimed is:

1. A method comprising:
administering to a subject suspected of being infected with a bacterium, a composition comprising:
a cobalt (III) compound having the formula CoR¹R²R³R⁴R⁵R⁶ or a salt thereof;
wherein each of R¹, R², R³, R⁴, and R⁵ is the same or different and includes an N-based ligand donor atom selected from the group consisting of ammonia, primary amine, or secondary amine, or salt thereof; and
wherein R⁶ is a ligand; and
an antibiotic compound.

2. The method of claim 1, wherein the cobalt (III) compound is Co(NH₃)₆³⁺ or a salt thereof.

3. The method of claim 1, wherein the cobalt (III) compound is CoCl(NH₃)₅²⁺ or a salt thereof

4. The method of claim 1, wherein the antibiotic is tetracycline.

5. The method of claim 1, wherein the antibiotic is streptomycin.

6. The method of claim 1, wherein the antibiotic is kanamycin.

7. The method of claim 1, wherein the antibiotic is gentamicin.

8. The method of claim 1, wherein the bacterium contains a TolC protein.

9. The method of claim 1, wherein the bacterium contains a CorA protein.

10. The method of claim 1, wherein the bacterium is Escherichia coli.

11. The method of claim 1, wherein the method produces an antibiotic effect greater than an equal amount of the antibiotic in the absence of the cobalt (III) compound.

12. The method of claim 1, wherein the subject has been diagnosed as needing a broad spectrum antibiotic.

13. A composition comprising:
a cobalt (III) compound having the formula CoR¹R²R³R⁴R⁵R⁶ or a salt thereof;
wherein each of R¹, R², R³, R⁴, and R⁵ is the same or different and includes an N-based ligand donor atom selected from the group consisting of ammonia, primary amine, or secondary amine, or salt thereof; and
wherein R⁶ is a ligand; and
an antibiotic compound.

14. The composition of claim 13, wherein the cobalt (III) compound is Co(NH₃)₆³⁺ or a salt thereof.

15. The composition of claim 13, wherein the cobalt (III) compound is CoCl(NH₃)₅²⁺ or a salt thereof.

16. The composition of claim 13, wherein the antibiotic is tetracycline.

17. The composition of claim 13, wherein the antibiotic is streptomycin.

18. The composition of claim 13, wherein the antibiotic is kanamycin.

19. The composition of claim 13, wherein the antibiotic is gentamicin.

20. A method comprising:
administering to a subject diagnosed as needing a broad spectrum antibiotic, a composition comprising:
a cobalt (III) compound having the formula CoR¹R²R³R⁴R⁵R⁶ or a salt thereof;
wherein each of R¹, R², R³, R⁴, and R⁵ is the same or different and includes an N-based ligand donor atom selected from the group consisting of ammonia, primary amine, or secondary amine, or salt thereof; and
wherein R⁶ is a ligand.

21. The method of claim 20, wherein the cobalt (III) compound is Co(NH₃)₆³⁺ or a salt thereof.
